(19) United States
(12) Patent Application Publication
(10) Pub. No.: US 2026/0306052 A1
(43) Pub. Date: Oct. 8, 2026

(54) ULTRASOUND DIAGNOSTIC APPARATUS, OPERATION METHOD OF ULTRASOUND DIAGNOSTIC APPARATUS, AND OPERATION PROGRAM OF ULTRASOUND DIAGNOSTIC APPARATUS

(71) Applicant: FUJIFILM Corporation, Tokyo (JP)

(72) Inventor: Yukiya MIYACHI, Kanagawa (JP)

(73) Assignee: FUJIFILM Corporation, Tokyo (JP)

(21) Appl. No.: 19/635,705

(22) Filed: Mar. 31, 2026

(30) Foreign Application Priority Data
Apr. 4, 2025 (JP) 2025-062407

(51) Int. Cl. A61B 8/08; A61B 8/00

(52) U.S. Cl. CPC A61B 8/085; A61B 8/461; A61B 8/587

(57) ABSTRACT

A selection unit selects a reference signal corresponding to a measurement target region from among a plurality of reference signals of a reference signal DB. A calculation unit calculates, based on a measurement signal obtained by measuring a patient and the selected reference signal, an attenuation rate of the measurement signal.

### CROSS-REFERENCE TO RELATED APPLICATIONS

The present application claims priority under 35 U.S.C. § 119 to Japanese Patent Application No. 2025-062407, filed on Apr. 4, 2025. The above application is hereby expressly incorporated by reference, in its entirety, into the present application.

### BACKGROUND

### 1. Technical Field

The technology of the present disclosure relates to an ultrasound diagnostic apparatus, an operation method of an ultrasound diagnostic apparatus, and an operation program of an ultrasound diagnostic apparatus.

A quantitative evaluation technology for liver fat using attenuation of ultrasound has attracted attention. In the quantitative evaluation technology, a reference method is mainly adopted due to high calculation accuracy of an attenuation rate. The reference method is a method of calculating the attenuation rate based on, for example, a reference signal obtained by measuring a phantom and a measurement signal obtained by actually measuring a subject (see Taku Kumada et al. “Quantitative evaluation of liver fat by ultrasound examination—Diagnosis of fatty liver from ‘subjective’ to ‘objective’-”, Innervision, Vol. 37, No. 3, March 2022, pp. 46 to 52).

### SUMMARY

In the measurement, a change in a measurement condition may be necessary, such as changing a measurement target region to a region avoiding a structure such as a blood vessel or a tissue boundary. In addition, in the quantitative evaluation technology for liver fat in the related art, the liver is mainly observed with a body surface probe, and a focus position, a frequency, and the like of a transmission wave are generally fixed. However, recently, there has been a demand for observing the liver with a body insertion probe or for observing a pancreas instead of the liver. In such a case, in a case where there is only one reference signal, the reference signal may not be suitable for the changed measurement condition, and the calculation accuracy of the attenuation rate may be decreased.

One embodiment according to the technology of the present disclosure provides an ultrasound diagnostic apparatus, an operation method of an ultrasound diagnostic apparatus, and an operation program of an ultrasound diagnostic apparatus capable of suppressing a decrease in calculation accuracy of an attenuation rate even in a case where a measurement condition is changed.

An ultrasound diagnostic apparatus according to the present disclosure comprises a processor, in which the processor is configured to select a reference signal corresponding to a measurement condition from among a plurality of reference signals, and calculate, based on a measurement signal obtained by measuring a subject and the selected reference signal, an attenuation rate of the measurement signal.

It is preferable that the measurement condition is at least one of a measurement target region, a focus position of a transmission wave, a gain applied to an echo signal, a frequency of the transmission wave, a type of a probe, or a measurement target organ.

It is preferable that the measurement condition is at least one of a measurement target region, a focus position of a transmission wave, a gain applied to an echo signal, or a frequency of the transmission wave.

It is preferable that the processor is configured to detect the measurement target region satisfying a detection condition, and select a reference signal corresponding to the detected measurement target region.

It is preferable that the detection condition includes a condition related to a distance to the measurement target organ and a condition related to an intensity of the echo signal.

It is preferable that the type of the probe is any of a body surface probe or a body insertion probe.

It is preferable that the measurement target organ is any of a liver or a pancreas.

It is preferable that calculation result information of the attenuation rate is displayed on a display unit.

It is preferable that the calculation result information is a color map in which a level of the attenuation rate is represented by a color.

It is preferable that the reference signal used for calculating the attenuation rate is displayed on the display unit together with the calculation result information.

It is preferable that the reference signal is any of a measurement signal obtained by using a phantom, a representative value of measurement signals of a plurality of representative subjects, or a measurement signal obtained by using a phantom, which is corrected by the representative value of the measurement signals of the plurality of representative subjects.

It is preferable that the reference signal is generated based on a transmission beam profile and a gain profile.

An operation method of an ultrasound diagnostic apparatus according to the present disclosure includes selecting a reference signal corresponding to a measurement condition from among a plurality of reference signals, and calculating, based on a measurement signal obtained by measuring a subject and the selected reference signal, an attenuation rate of the measurement signal.

An operation program of an ultrasound diagnostic apparatus according to the present disclosure causes a computer to execute a process including selecting a reference signal corresponding to a measurement condition from among a plurality of reference signals, and calculating, based on a measurement signal obtained by measuring a subject and the selected reference signal, an attenuation rate of the measurement signal.

According to the technology of the present disclosure, it is possible to provide an ultrasound diagnostic apparatus, an operation method of an ultrasound diagnostic apparatus, and an operation program of an ultrasound diagnostic apparatus capable of suppressing a decrease in calculation accuracy of an attenuation rate even in a case where a measurement condition is changed.

### BRIEF DESCRIPTION OF THE DRAWINGS

FIG. 1 is a diagram showing a state of ultrasound diagnosis using a body surface probe.

FIG. 2 is a block diagram showing an electric configuration of an ultrasound diagnostic apparatus.

FIG. 3 is a block diagram showing a detailed configuration of a controller.

FIG. 4 is a block diagram showing a processing unit of a CPU.

FIG. 5 is a diagram showing a reference signal DB.

FIG. 6 is a diagram showing a setting information table.

FIG. 7 is a diagram showing a state in which a measurement target region is changed to avoid a blood vessel.

FIG. 8 is a diagram showing an aspect of selecting a reference signal corresponding to the measurement target region.

FIG. 9 is a diagram showing an aspect of selecting setting information corresponding to a measurement target region.

FIG. 10 is a diagram showing an attenuation rate in a case where Equation (2) is used.

FIG. 11 is a diagram showing a diagnosis screen.

FIG. 12 is a diagram showing a processing procedure of the ultrasound diagnostic apparatus.

FIG. 13 is a diagram showing a second embodiment in which a measurement target region satisfying a detection condition is detected and a reference signal corresponding to the detected measurement target region is selected.

FIG. 14 is a diagram showing an aspect of selecting a reference signal corresponding to a focus position.

FIG. 15 is a diagram showing an aspect of selecting a reference signal corresponding to a gain.

FIG. 16 is a diagram showing an aspect of selecting a reference signal corresponding to a frequency.

FIG. 17 is a diagram showing a state of ultrasound diagnosis using a body insertion probe.

FIG. 18 is a specification table of a body surface probe and a body insertion probe.

FIG. 19 is a specification table of the body surface probe and the body insertion probe.

FIG. 20 is a diagram showing an aspect of selecting a reference signal corresponding to a probe.

FIG. 21 is a diagram showing an example in which a pancreas is used as a measurement target organ.

FIG. 22 is a diagram showing an aspect of selecting a reference signal corresponding to a measurement target organ.

FIG. 23 is a diagram showing an aspect of displaying a color map of an attenuation rate.

FIG. 24 is a diagram showing an example in which a measurement signal obtained by using a phantom is used as a reference signal.

FIG. 25 is a diagram showing an example in which an average value of measurement signals of a plurality of representative patients is used as a reference signal.

FIG. 26 is a diagram showing an example in which a measurement signal obtained by using a phantom, which is corrected by an average value of measurement signals of a plurality of representative patients, is used as a reference signal.

FIG. 27 is a diagram showing an example in which a reference signal is generated by adding a transmission beam profile and a gain profile.

### DETAILED DESCRIPTION

### First Embodiment

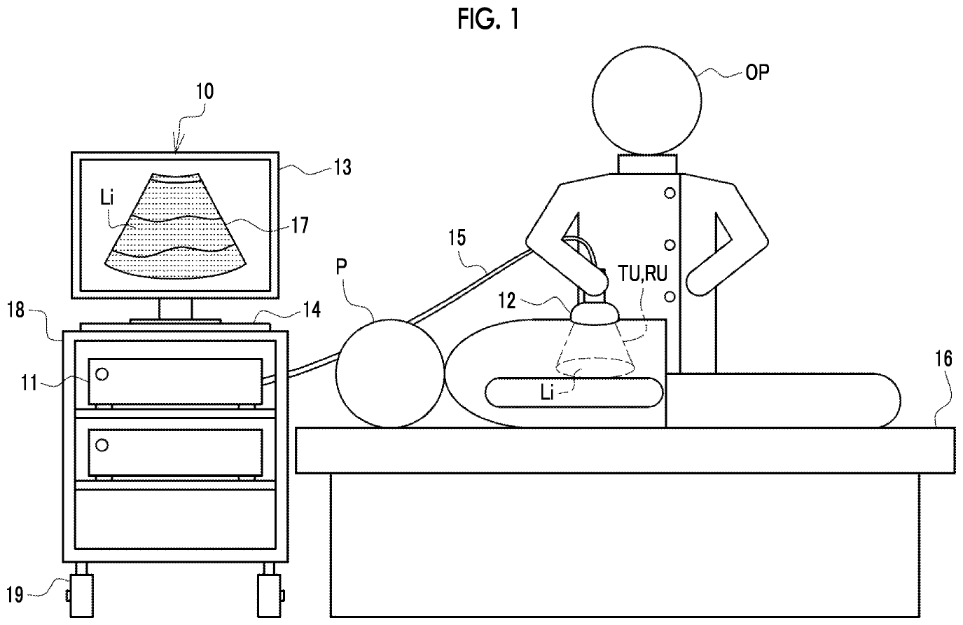

As shown in FIG. 1 as an example, an ultrasound diagnostic apparatus 10 comprises an apparatus body 11, a body surface probe 12, a display 13, and an input device 14. The apparatus body 11 and the body surface probe 12 are connected by a cable 15. The body surface probe 12 is gripped by an operator OP such as an ultrasound technician, and a distal end thereof is placed on an abdominal surface of a patient P lying on an examination table 16. A transmission wave TU is transmitted from the distal end of the body surface probe 12. The transmission wave TU reaches a liver Li. The patient P is an example of a “subject” according to the technology of the present disclosure. The liver Li is an example of a “measurement target organ” according to the technology of the present disclosure.

The display 13 displays an ultrasound image based on a reflected wave RU reflected in a body of the patient P in response to the transmission wave TU, here, a brightness (B) mode image 17. For example, a subcutaneous tissue and the liver Li are shown in the B-mode image 17. In addition, the display 13 displays various screens such as a registration screen of the patient P and a setting screen of a focus position of the transmission wave TU. The various screens have an operation function by a graphical user interface (GUI). The apparatus body 11 receives an input of an operation instruction from an input device 14 through the various screens. The input device 14 is a keyboard, a mouse, a touch panel, a microphone for voice input, or the like. The display 13 is an example of a “display unit” according to the technology of the present disclosure.

The apparatus body 11, the display 13, and the input device 14 are mounted on a cart 18. The cart 18 has wheels 19 and is movable in a medical facility.

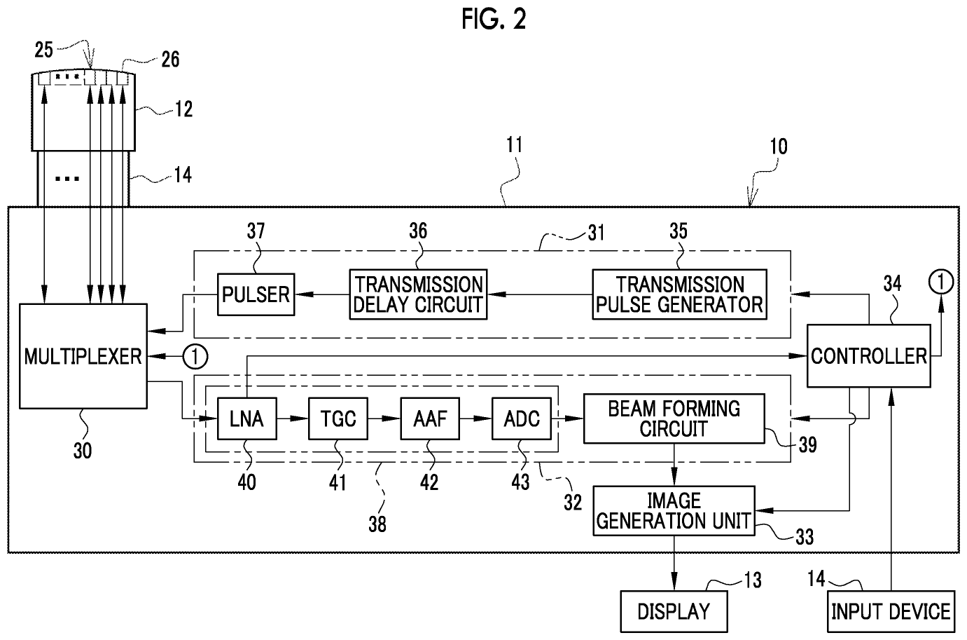

As shown in FIG. 2 as an example, the body surface probe 12 has a transducer array 25 built in a distal end thereof. The transducer array 25 is composed of a plurality of (for example, 64 to 192) aligned transducers 26. The transducer array 25 transmits a transmission wave TU and receives a reflected wave RU. The transducer array 25 includes, for example, a convex type, a linear type, and a sector type. In the convex type, the transducers 26 are arranged in a curved shape to form a fan-shaped transmission wave TU. In the linear type, the transducers 26 are arranged in a linear shape to form a rectangular transmission wave TU. In the sector type, the transducers 26 are densely arranged in a narrow opening portion, and a fan-shaped transmission wave TU is formed by electronic scanning. In the present example, the convex type is suitable for observing an abdomen of the patient P. In addition to the transducer array 25, an acoustic lens or the like is built in the distal end of the body surface probe 12.

The apparatus body 11 comprises a multiplexer 30, a transmission unit 31, a reception unit 32, an image generation unit 33, and a controller 34. The transducer 26 is connected to the multiplexer 30. In addition, the transmission unit 31 and the reception unit 32 are connected to the multiplexer 30. The multiplexer 30 is responsible for transmission control of the transmission wave TU and reception control of the reflected wave RU. The transmission control of the transmission wave TU is control of generating the transmission wave TU from the selected transducer 26 by transmitting a transmission pulse to the selected transducer 26 among the plurality of transducers 26. The reception control of the reflected wave RU is control of distributing an electric signal (hereinafter, referred to as an echo signal) corresponding to the reflected wave RU received by each transducer 26 to an appropriate channel of the reception unit 32.

The transmission unit 31 includes a transmission pulse generator 35, a transmission delay circuit 36, and a pulser 37. The transmission pulse generator 35 generates a transmission pulse having a set frequency and pulse width, and outputs the transmission pulse to the transmission delay circuit 36. The transmission delay circuit 36 adjusts a focus position of the transmission wave TU by controlling a supply timing of the transmission pulse to each transducer 26. Specifically, the transmission delay circuit 36 adjusts the focus position to a relatively shallow position by supplying the transmission pulse to the transducer 26 of a central portion first and then supplying the transmission pulse to the transducer 26 of an end portion. On the other hand, the transmission delay circuit 36 adjusts the focus position to a relatively deep position by supplying the transmission pulse to the transducer 26 of the end portion first and then supplying the transmission pulse to the transducer 26 of the central portion. The pulser 37 supplies the transmission pulse to the transducer 26 through the multiplexer 30.

The reception unit 32 includes an analog front end circuit 38 and a beam forming circuit 39. The analog front end circuit 38 includes a low-noise amplifier (hereinafter, referred to as a low noise amplifier (LNA)) 40, a time gain correction amplifier (hereinafter, referred to as a time gain compensation (TGC)) 41, a fold-back noise prevention filter (hereinafter, referred to as an anti-aliasing filter (AAF)) 42, and an analog/digital converter (hereinafter, referred to as an analog-to-digital converter (ADC)) 43. Although the analog front end circuit 38 is drawn for only one channel in FIG. 2, there are actually a plurality of channels (for example, 16 channels to 256 channels).

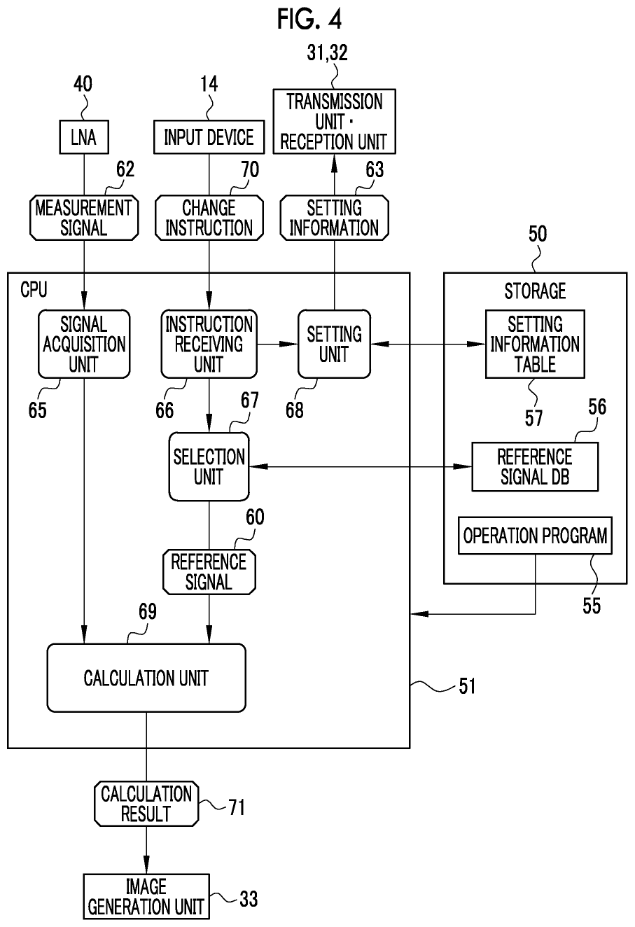

The LNA 40 amplifies an echo signal corresponding to the reflected wave RU from the transducer 26 to a set level. The LNA 40 outputs the amplified echo signal to the TGC 41 and the controller 34. Hereinafter, the echo signal output from the LNA 40 to the controller 34 is referred to as a measurement signal 62 (see FIG. 4).

The TGC 41 applies a gain corresponding to a propagation distance to the echo signal in order to correct attenuation of the echo signal due to the propagation distance. In other words, the TGC 41 emphasizes the echo signal of a deep portion (distal portion) as compared with the echo signal of a shallow portion (proximal portion). As a result, the brightness and contrast of the ultrasound image such as the B-mode image 17 are homogenized. The TGC 41 outputs the analog echo signal after the time gain correction to the AAF 42. The gain applied to the echo signal in the TGC 41 is an example of a “gain applied to an echo signal” according to the technology of the present disclosure.

The AAF 42 is a so-called low-pass filter, and prevents the occurrence of fold-back noise (aliasing) by removing high-frequency components exceeding a Nyquist frequency from the echo signal. The ADC 43 converts the analog echo signal that has passed through the AAF 42 into a digital echo signal, and outputs the digital echo signal to the beam forming circuit 39. The beam forming circuit 39 corrects a time difference between the respective echo signals from the analog front end circuit 38 and then adds the echo signals. As a result, the beam forming circuit 39 obtains beam forming data necessary for generating the ultrasound image. The beam forming data includes amplitude information and phase information of each echo signal. The beam forming circuit 39 outputs the beam forming data to the image generation unit 33.

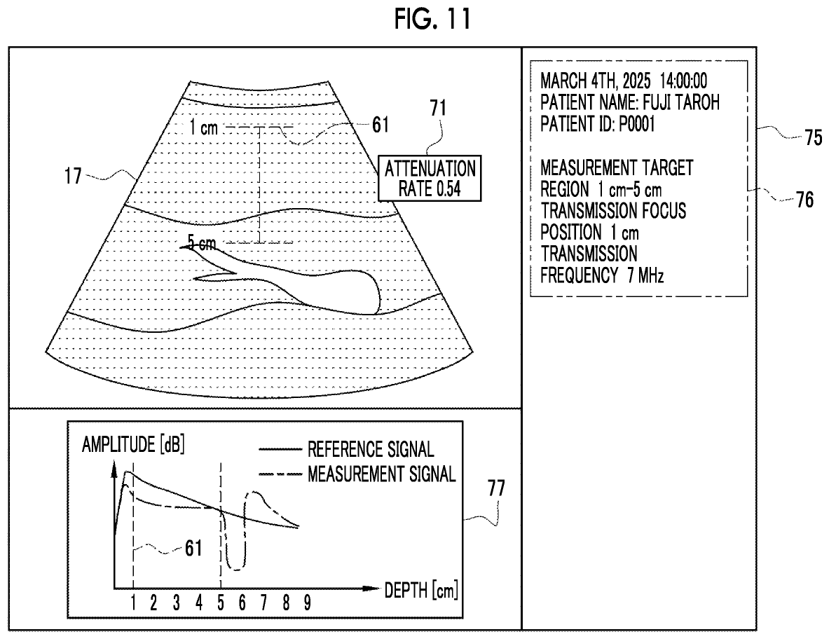

The image generation unit 33 performs digital time gain correction on the beam forming data in accordance with the gain of the setting information 63. The image generation unit 33 generates the ultrasound image based on the beam forming data. The image generation unit 33 can generate a motion (M) mode image, a Doppler image, or the like as the ultrasound image in addition to the illustrated B-mode image 17. The image generation unit 33 constitutes a diagnosis screen 75 (see FIG. 11) including the generated ultrasound image, and displays the diagnosis screen 75 on the display 13.

The controller 34 comprehensively controls the operations of the respective units such as the multiplexer 30, the transmission unit 31, the reception unit 32, and the image generation unit 33. Specifically, the controller 34 connects each transducer 26 to the transmission unit 31 and the reception unit 32 via the multiplexer 30. The controller 34 sets the focus position and the frequency in the transmission unit 31, and adjusts a generation timing and a waveform of the transmission pulse. The controller 34 sets the gain in the TGC 41 of the reception unit 32, and sets the beam forming condition such as the delay time in the beam forming circuit 39. In addition, the controller 34 causes the image generation unit 33 to generate the ultrasound image in accordance with the selected display mode.

Various operation instructions from the operator OP are input to the controller 34 through the input device 14. The various operation instructions include a switching instruction of the display mode of the ultrasound image, and the like in addition to a transmission instruction of the transmission wave TU. In addition, the various operation instructions include a start instruction and an end instruction of attenuation rate calculation processing of the liver Li, which will be described below, and a change instruction 70 (see FIG. 4) of a measurement target region of the attenuation rate to be set in the liver Li. The controller 34 performs control in accordance with the various operation instructions. A switch for giving the transmission instruction of the transmission wave TU and/or the start instruction and the end instruction of the attenuation rate calculation processing of the liver Li may be provided in the body surface probe 12.

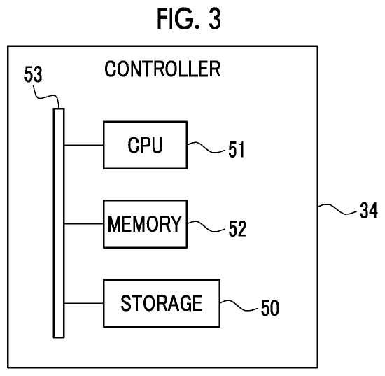

As shown in FIG. 3 as an example, the controller 34 comprises a storage 50, a central processing unit (CPU) 51, and a memory 52. The storage 50, the CPU 51, and the memory 52 are connected to each other through a busline 53. The controller 34 is an example of a “computer” according to the technology of the present disclosure.

The storage 50 is, for example, a hard disk drive. The storage 50 stores various programs, various types of data associated with the various programs, and the like. Note that a solid-state drive may be used instead of the hard disk drive.

The memory 52 is a work memory for executing processing via the CPU 51. The CPU 51 loads a program stored in the storage 50 into the memory 52 and executes processing corresponding to the program. As a result, the CPU 51 comprehensively controls each unit of the apparatus body 11. The CPU 51 is an example of a “processor” according to the technology of the present disclosure. The memory 52 may be built in the CPU 51.

As shown in FIG. 4 as an example, the storage 50 stores an operation program 55. The operation program 55 is a program for causing the CPU 51 to perform attenuation rate calculation processing and the like. That is, the operation program 55 is an example of an “operation program of an ultrasound diagnostic apparatus” according to the technology of the present disclosure. A reference signal database (hereinafter, referred to as a database (DB)) 56 and a setting information table 57 are also stored in the storage 50.

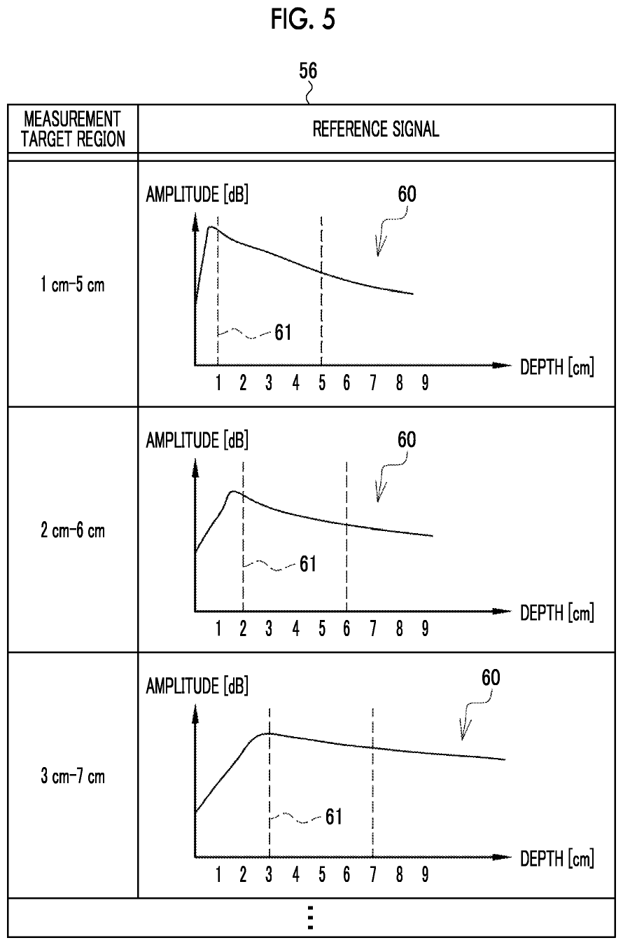

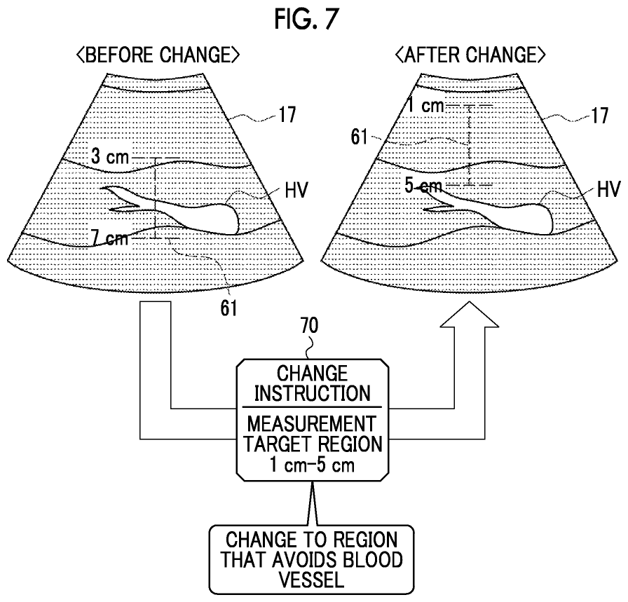

As shown in FIG. 5 as an example, a reference signal 60 is registered in the reference signal DB 56 for each measurement target region. The measurement target region is represented by a distance in a depth direction from a body surface, and is, for example, 1 cm to 5 cm, 2 cm to 6 cm, 3 cm to 7 cm, or the like. The measurement target region can be appropriately changed by the operator OP (see FIG. 7). The measurement target region is an example of a “measurement condition” according to the technology of the present disclosure.

The reference signal 60 has an amplitude that rapidly increases in the vicinity of the focus position in front of the measurement target region indicated by a broken line 61, and then decreases. Then, the amplitude gradually decreases in the measurement target region. In addition, in the reference signal 60, a decrease rate of the amplitude is smaller as the measurement target region is deeper.

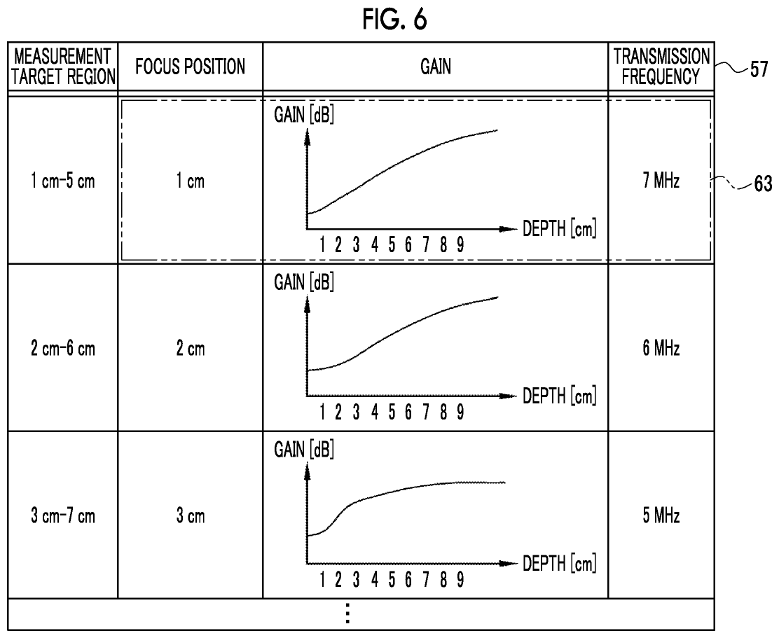

As shown in FIG. 6 as an example, in the setting information table 57, the focus position of the transmission wave TU, the gain applied to the echo signal, and the frequency of the transmission wave TU are registered for each measurement target region. Numerical values actually set in the transmission unit 31 are registered in the focus position and the frequency. Numerical values of the gain actually set in the reception unit 32 at each depth are registered in the gain.

For example, a shallow portion position of the measurement target region is set as the focus position. Specifically, the focus position is 1 cm in a case where the measurement target region is 1 cm to 5 cm, 2 cm in a case where the measurement target region is 2 cm to 6 cm, and 3 cm in a case where the measurement target region is 3 cm to 7 cm. That is, the focus position is also set deeper as the measurement target region is deeper. By appropriately controlling the transmission delay of the transmission wave, the focus position having a certain depth width may be set, such as 0.5 cm to 1.5 cm in a case where the measurement target region is 1 cm to 5 cm, 1.5 cm to 2.5 cm in a case where the measurement target region is 2 cm to 6 cm, and 2.5 cm to 3.5 cm in a case where the measurement target region is 3 cm to 7 cm.

The gain is set such that the value of the deep portion is higher than the value of the shallow portion, regardless of the measurement target region. In addition, the gain is set such that the value of a shallowest portion close to the body surface of 1 cm to 3 cm is lower as the measurement target region is shallower. The reason why the value of the shallowest portion close to the body surface is set to be low is to prevent the saturation of the echo signal in the shallowest portion.

The frequency is set to a lower value as the measurement target region is deeper. Specifically, the frequency is 7 MHz in a case where the measurement target region is 1 cm to 5 cm, 6 MHz in a case where the measurement target region is 2 cm to 6 cm, and 5 MHz in a case where the measurement target region is 3 cm to 7 cm.

As described above, the focus position, the gain, and the frequency are set to values corresponding to the measurement target region. The focus position, the gain, and the frequency are also an example of a “measurement condition” according to the technology of the present disclosure. It should be noted that, hereinafter, the set of the focus position, the gain, and the frequency will be collectively referred to as setting information 63.

In a case where the start instruction of the attenuation rate calculation processing is input by the operator OP through the input device 14, the operation program 55 is activated. In a case where the operation program 55 is activated, as shown in FIG. 4, the CPU 51 functions as a signal acquisition unit 65, an instruction receiving unit 66, a selection unit 67, a setting unit 68, and a calculation unit 69 in cooperation with the memory 52 and the like. The signal acquisition unit 65, the instruction receiving unit 66, the selection unit 67, the setting unit 68, and the calculation unit 69, particularly the selection unit 67 and the calculation unit 69, execute the attenuation rate calculation processing. It should be noted that the CPU 51 functions as various processing units in addition to each of the processing units.

The signal acquisition unit 65 acquires the measurement signal 62 from the LNA 40. The signal acquisition unit 65 outputs the measurement signal 62 to the calculation unit 69. In addition, although not shown, the signal acquisition unit 65 outputs the measurement signal 62 to the image generation unit 33.

The instruction receiving unit 66 receives various operation instructions input by the operator OP through the input device 14. In particular, the instruction receiving unit 66 receives a change instruction 70 for the measurement target region of the attenuation rate. The instruction receiving unit 66 outputs the change instruction 70 to the selection unit 67 and the setting unit 68.

The selection unit 67 reads out the reference signal 60 corresponding to the measurement target region from among the plurality of reference signals 60 registered in the reference signal DB 56. The selection unit 67 outputs the readout reference signal 60 to the calculation unit 69. In addition, although not shown, the selection unit 67 outputs the readout reference signal 60 to the image generation unit 33.

The setting unit 68 reads out the setting information 63 corresponding to the measurement target region of the change instruction 70 from among the plurality of pieces of setting information 63 registered in the setting information table 57. The setting unit 68 outputs the focus position and the frequency among the readout pieces of setting information 63 to the transmission unit 31, and outputs the gain to the reception unit 32.

In the initial default setting in which the start instruction for the attenuation rate calculation processing is given, the selection unit 67 reads out the reference signal 60 corresponding to the measurement target region of 3 cm to 7 cm from the reference signal DB 56. Similarly, in the default setting, the setting unit 68 also reads out the setting information 63 corresponding to the measurement target region of 3 cm to 7 cm from the setting information table 57.

As shown in FIG. 7 as an example, in a case where a blood vessel such as a portal vein, a hepatic vein, or a hepatic artery, which may hinder the accurate calculation of the attenuation rate, is present in the current measurement target region, the operator OP gives the change instruction 70 to the measurement target region that avoids the blood vessel. FIG. 7 shows an example in which the measurement target region is changed from 3 cm to 7 cm to 1 cm to 5 cm in order to avoid the hepatic vein HV.

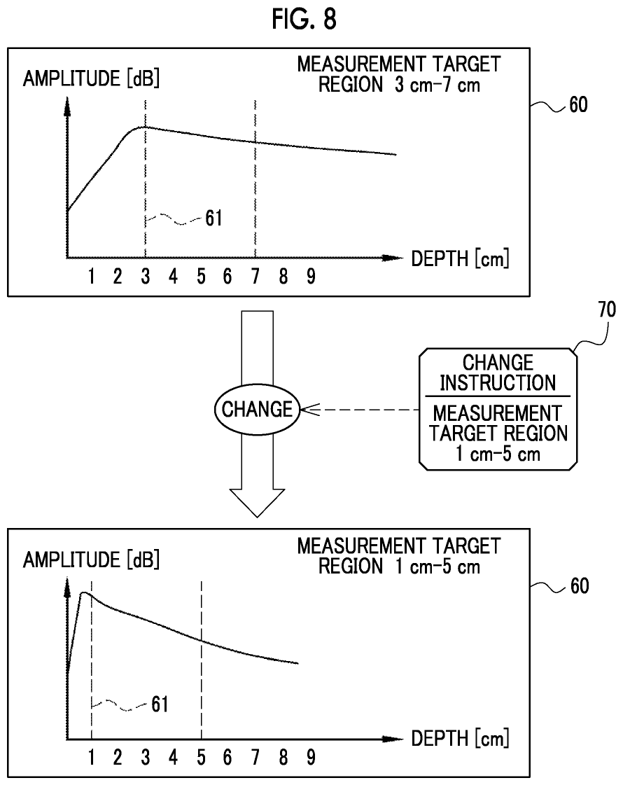

In such a case, as shown in FIG. 8 as an example, the selection unit 67 reads out the reference signal 60 corresponding to the measurement target region of the change instruction 70, here, the measurement target region of 1 cm to 5 cm, from the reference signal DB 56, and outputs the readout reference signal 60 to the calculation unit 69. In other words, the selection unit 67 selects the reference signal 60 corresponding to the measurement target region of 1 cm to 5 cm instead of the reference signal 60 corresponding to the measurement target region of 3 cm to 7 cm.

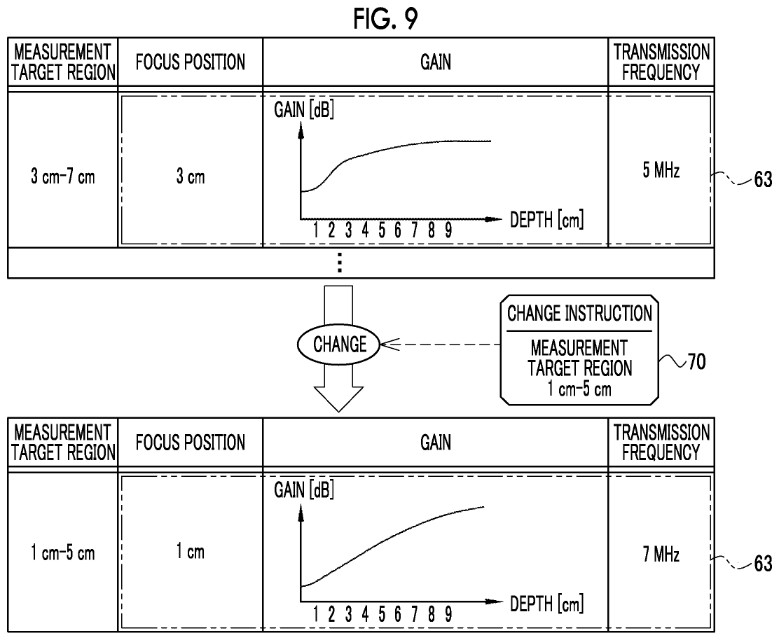

In addition, as shown in FIG. 9 as an example, the setting unit 68 reads out the setting information 63 corresponding to the measurement target region of the change instruction 70, here, the measurement target region of 1 cm to 5 cm, from the setting information table 57, and outputs the readout setting information 63 to the transmission unit 31 and the reception unit 32. In other words, the setting unit 68 sets the setting information 63 corresponding to the measurement target region of 1 cm to 5 cm in the transmission unit 31 and the reception unit 32 instead of the setting information 63 corresponding to the measurement target region of 3 cm to 7 cm. Since the setting information 63 corresponding to the measurement target region of the change instruction 70 is set in this way, as a result, the reference signal 60 corresponding to not only the measurement target region, but also to the focus position of the transmission wave TU, the gain applied to the echo signal, and the frequency of the transmission wave TU, is selected.

It should be noted that the blood vessel exemplified as the factor that may hinder the calculation of the attenuation rate is not limited thereto. For example, various artifacts such as multiple reflection (reverberation artifact), echo from a side lobe (side lobe artifact), or refraction (refraction artifact) may also hinder the accurate calculation of the attenuation rate.

In FIG. 4, the calculation unit 69 calculates the attenuation rate of the measurement signal 62 based on the measurement signal 62 from the signal acquisition unit 65 and the reference signal 60 from the selection unit 67. The calculation unit 69 outputs a calculation result 71 of the attenuation rate to the image generation unit 33. The calculation result 71 is an example of “calculation result information” according to the technology of the present disclosure.

The unit of the attenuation rate is dB/cm/MHz. In a case where the attenuation rate is 0.6 dB/cm/MHz or less, it is diagnosed as normal (no fat deposition), and in a case where the attenuation rate is 0.7 dB/cm/MHz or more, it is diagnosed as having a high likelihood of fatty liver. In particular, in a case where the attenuation rate is 1.0 dB/cm/MHz or more, it is diagnosed as severe fatty liver in which the risk of liver dysfunction such as cirrhosis should also be considered.

In a case where the attenuation rate of the reference signal 60 is denoted by αref, the depth of the shallow portion is denoted by d1, the depth of the deep portion is denoted by d2, the amplitude of the measurement signal 62 is denoted by Pf(d), the amplitude of the reference signal 60 is denoted by Pref(d), and the frequency is denoted by f, the calculation unit 69 calculates the attenuation rate a from the following Equation (1).

$$α = \frac{- \{ Pf ( d 2 ) - Pref ( d 2 ) \} + \{ Pf ( d 1 ) - Pref ( d 1 ) \}}{2 ( d 2 - d 1 ) \cdot f} + α ref \qquad ( 1 )$$

Alternatively, the attenuation rate a may be simply calculated from the following Equation (2).

$$α = \frac{- \{ Pf ( d 2 ) - Pref ( d 2 ) \} + \{ Pf ( d 1 ) - Pref ( d 1 ) \}}{( d 2 - d 1 )} \qquad ( 2 )$$

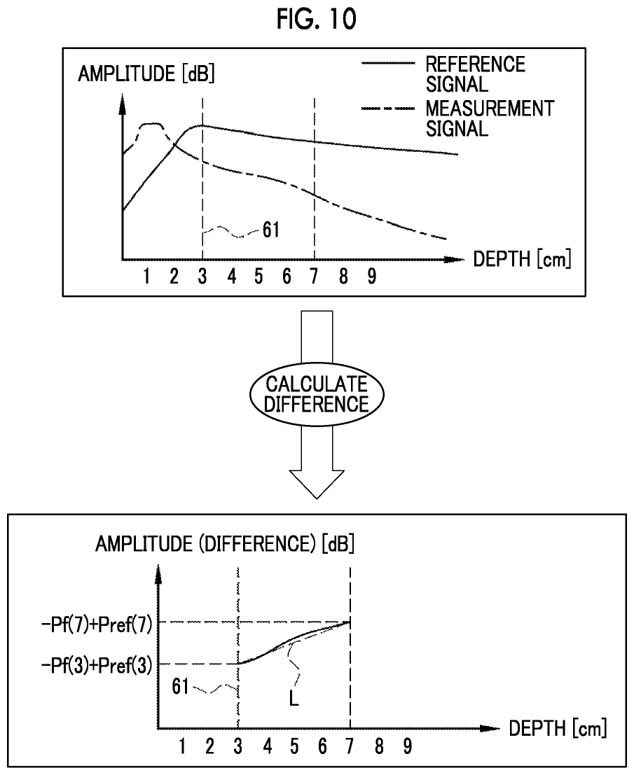

As shown in FIG. 10 as an example, in a case where Equation (2) is used, the attenuation rate a is the slope of a straight line L connecting the difference Pf(d1)−Pref(d1) in the amplitudes of the measurement signal 62 and the reference signal 60 at the depth d1 and the difference Pf(d2)−Pref(d2) in the amplitudes of the measurement signal 62 and the reference signal 60 at the depth d2. That is, Equation (2) is an equation in which the attenuation rate is linearly approximated by using the difference in the amplitudes of the measurement signal 62 and the reference signal 60 at the depths d1 and d2. In FIG. 10, d1=3 cm and d2=7 cm.

As shown in FIG. 11 as an example, a diagnosis screen 75 displays basic measurement information 76 such as the measurement date and time, the patient information, the measurement target region, the focus position, and the frequency. In addition, the B-mode image 17 and the calculation result 71 of the attenuation rate are displayed on the diagnosis screen 75. A broken line 61 indicating the measurement target region and numerical values indicating the depths of the shallow portion and the deep portion of the measurement target region are displayed in a superimposed manner on the B-mode image 17. Further, a graph 77 showing the amplitudes of the measurement signal 62 and the reference signal 60 used for calculating the attenuation rate with respect to the depth is displayed on the diagnosis screen 75. The operator OP diagnoses whether or not there is a sign of fatty liver in the liver Li of the patient P while viewing the diagnosis screen 75.

Next, the actions obtained from the above-described configuration will be described. First, the patient P lies on the examination table 16 by the guidance of the operator OP. The operator OP turns on the power of the ultrasound diagnostic apparatus 10 to display a registration screen of the patient P on the display 13 and inputs the patient information through the input device 14.

The operator OP grips the body surface probe 12, places the body surface probe 12 on the abdominal surface of the patient P, and inputs a transmission instruction of the transmission wave TU through the input device 14. As a result, the transmission unit 31 and the multiplexer 30 are driven under the control of the controller 34, and the transmission wave TU is transmitted from the transducer array 25.

The transmission wave TU propagates in the body of the patient P, and a part of the transmission wave TU is reflected due to an acoustic impedance difference between tissues. The reflected wave RU generated in this way is received by the transducer array 25 and is converted into an echo signal. The echo signal is digitized through the multiplexer 30 and the analog front end circuit 38 under the control of the controller 34, and is input to the beam forming circuit 39. In the beam forming circuit 39, the echo signal is subjected to delay correction and addition processing, and beam forming data is generated. The beam forming data is output from the beam forming circuit 39 to the image generation unit 33.

In the image generation unit 33, an ultrasound image, for example, the B-mode image 17, is generated based on the beam forming data. Then, the diagnosis screen 75 including the B-mode image 17 is configured and is displayed on the display 13.

The operator OP adjusts a position where the body surface probe 12 is placed to a position suitable for observing the liver Li while viewing the B-mode image 17 on the diagnosis screen 75. Thereafter, the operator OP inputs a start instruction of the attenuation rate calculation processing of the liver Li through the input device 14. As a result, the operation program 55 is activated, and the CPU 51 of the controller 34 functions as a signal acquisition unit 65, an instruction receiving unit 66, a selection unit 67, a setting unit 68, and a calculation unit 69 as shown in FIG. 4.

In the default setting, the reference signal 60 corresponding to the measurement target region of 3 cm to 7 cm is read out from the reference signal DB 56 by the selection unit 67 and is output to the calculation unit 69 and the image generation unit 33. Similarly, the setting unit 68 reads out the setting information 63 corresponding to the measurement target region of 3 cm to 7 cm from the setting information table 57 and outputs the setting information 63 to the transmission unit 31 and the reception unit 32.

A measurement signal 62 is input to the signal acquisition unit 65 from the LNA 40. The measurement signal 62 is output from the signal acquisition unit 65 to the calculation unit 69 and the image generation unit 33.

The calculation unit 69 calculates the attenuation rate based on the measurement signal 62 and the reference signal 60. A calculation result 71 of the attenuation rate is output from the calculation unit 69 to the image generation unit 33.

As shown in FIG. 11, the image generation unit 33 displays the calculation result 71 and a graph 77 of the measurement signal 62 and the reference signal 60 used for calculating the attenuation rate on the diagnosis screen 75.

As shown in FIG. 7, in a case where the hepatic vein HV or the like that may hinder the accurate calculation of the attenuation rate is present in the current measurement target region, the operator OP inputs a change instruction 70 through the input device 14.

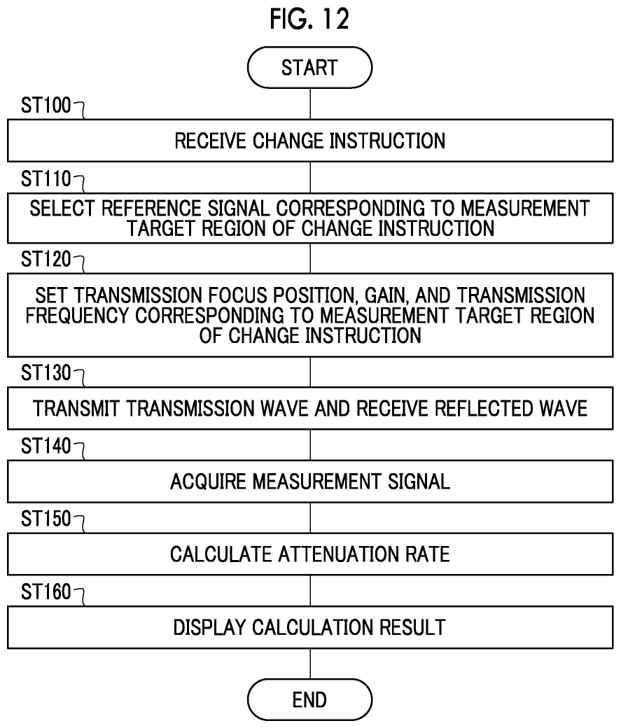

As shown in FIG. 12 as an example, the change instruction 70 is received by the instruction receiving unit 66 (step ST100). The change instruction 70 is output from the instruction receiving unit 66 to the selection unit 67 and the setting unit 68.

As shown in FIG. 8, the selection unit 67 reads out the reference signal 60 corresponding to the measurement target region of the change instruction 70 from the reference signal DB 56. The readout reference signal 60 is output from the selection unit 67 to the calculation unit 69. In this way, the reference signal 60 corresponding to the measurement target region of the change instruction 70 is selected (step ST110).

In addition, as shown in FIG. 9, the setting unit 68 reads out the setting information 63 corresponding to the measurement target region of the change instruction 70 from the setting information table 57. The readout setting information 63 is output from the setting unit 68 to the transmission unit 31 and the reception unit 32. In this way, the setting information 63 corresponding to the measurement target region of the change instruction 70 is set in the transmission unit 31 and the reception unit 32 (step ST120).

The transmission wave TU corresponding to the focus position and the frequency of the setting information 63 is transmitted from the transducer array 25, and the reflected wave RU generated by this is received by the transducer array 25 (step ST130). The echo signal corresponding to the reflected wave RU is amplified to the set level by the LNA 40 and then output from the LNA 40 to the controller 34 as the measurement signal 62. In addition, the gain of the setting information 63 is applied to the echo signal by the TGC 41, and the echo signal is digitized by the ADC 43. The digitized echo signal is converted into beam forming data by the beam forming circuit 39. The beam forming data is subjected to digital time gain correction corresponding to the gain of the setting information 63 in the image generation unit 33. Then, the image generation unit 33 generates the B-mode image 17 based on the beam forming data, and displays the B-mode image 17 on the diagnosis screen 75.

The measurement signal 62 is acquired by the signal acquisition unit 65 (step ST140). The measurement signal 62 is output to the calculation unit 69, and the calculation unit 69 calculates the attenuation rate based on the measurement signal 62 and the reference signal 60 (step ST150). The calculation result 71 of the attenuation rate is output to the image generation unit 33 and is displayed on the diagnosis screen 75 as shown in FIG. 11 (step ST160).

As described above, the CPU 51 of the controller 34 functions as the selection unit 67 and the calculation unit 69. The selection unit 67 selects the reference signal 60 corresponding to the measurement target region from among the plurality of reference signals 60 of the reference signal DB 56. The calculation unit 69 calculates the attenuation rate of the measurement signal 62 based on the measurement signal 62 obtained by measuring the patient P and the selected reference signal 60. The reference signal 60 selected by the selection unit 67 is the reference signal 60 suitable for calculating the attenuation rate of the measurement signal 62 in the measurement target region. Therefore, it is possible to suppress a decrease in calculation accuracy of the attenuation rate even in a case where the measurement target region is changed.

In the present first embodiment, the measurement condition is the measurement target region. In addition, as shown in FIG. 9, the measurement condition further includes the focus position of the transmission wave TU, the gain applied to the echo signal, and the frequency of the transmission wave TU. Therefore, it is possible to suppress a decrease in calculation accuracy of the attenuation rate even in a case where the measurement target region, the focus position of the transmission wave TU, the gain applied to the echo signal, and the frequency of the transmission wave TU are changed.

As shown in FIG. 11, the calculation result 71 of the attenuation rate is displayed on the display 13. Therefore, the operator OP can check the calculation result 71 of the attenuation rate in real time. The operator OP can quickly diagnose whether or not the liver Li of the patient P has a sign of fatty liver. In addition, since the decrease in calculation accuracy of the attenuation rate is suppressed, it is possible to make a diagnosis with high accuracy.

As shown in FIG. 11, the reference signal 60 used for calculating the attenuation rate is displayed on the display 13 together with the calculation result 71. Therefore, the operator OP can check which reference signal 60 is actually used to calculate the attenuation rate, and can verify the validity of the diagnosis.

### Second Embodiment

In the first embodiment, a case where the operator OP inputs the change instruction 70 for the measurement target region has been described as an example, but the present disclosure is not limited thereto.

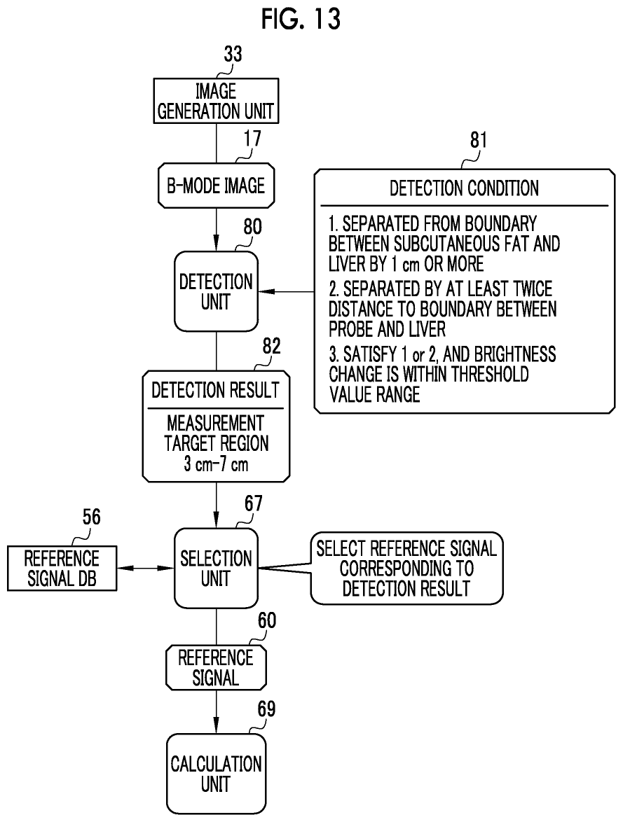

As shown in FIG. 13 as an example, the CPU 51 according to the present second embodiment functions as a detection unit 80 in addition to the respective processing units 65 to 69 (not shown in FIG. 13 except for the selection unit 67 and the calculation unit 69) according to the first embodiment. The B-mode image 17 is input to the detection unit 80 from the image generation unit 33. In addition, a detection condition 81 is input to the detection unit 80. The detection condition 81 is stored in the storage 50.

The detection condition 81 is a condition for detecting the measurement target region that is considered not to hinder the calculation of the attenuation rate. The detection condition 81 includes, in a broad sense, a condition related to a distance to the measurement target organ, here, the liver Li, and a condition related to an intensity of the echo signal. The first condition related to the distance to the measurement target organ is that the measurement target region is separated from a boundary between subcutaneous fat and the liver Li by 1 cm or more. The second condition related to the distance to the measurement target organ is that the measurement target region is separated by at least twice a distance to a boundary between the body surface probe 12 and the liver Li. These conditions are based on the fact that the disturbance of the measurement signal 62 is large in the vicinity of the boundary between subcutaneous fat and the liver Li, and it is difficult to accurately calculate the attenuation rate.

The condition related to the intensity of the echo signal is that any one of the first or second condition related to the distance to the measurement target organ is satisfied, and a change in brightness corresponding to the intensity of the echo signal of the B-mode image 17 is within a threshold value range. A range in which it is considered that there are no blood vessels and various artifacts and that a parenchyma of the liver Li is uniformly present is set as the threshold value range.

The detection unit 80 performs image analysis on the B-mode image 17 to detect the measurement target region that satisfies the detection condition 81. The detection unit 80 outputs a detection result 82 of the measurement target region to the selection unit 67. The selection unit 67 reads out the reference signal 60 corresponding to the detection result 82 from the reference signal DB 56, and outputs the readout reference signal 60 to the calculation unit 69.

As described above, in the second embodiment, the detection unit 80 detects the measurement target region that satisfies the detection condition 81, and the selection unit 67 selects the reference signal 60 corresponding to the detection result 82. Therefore, it is possible to save the time and effort of the operator OP to input the change instruction 70 for the measurement target region. In addition, it is possible to always acquire the measurement signal 62 of the measurement target region that is considered not to hinder the calculation of the attenuation rate. It should be noted that the B-mode image 17 may be input to a trained model, and the measurement target region that is considered not to hinder the calculation of the attenuation rate may be output from the trained model.

The detection condition 81 includes a condition related to the distance of the measurement target organ and a condition related to the intensity of the echo signal. Therefore, it is possible to detect the measurement target region that is considered not to hinder the calculation of the attenuation rate easily and with high accuracy.

### Third Embodiment

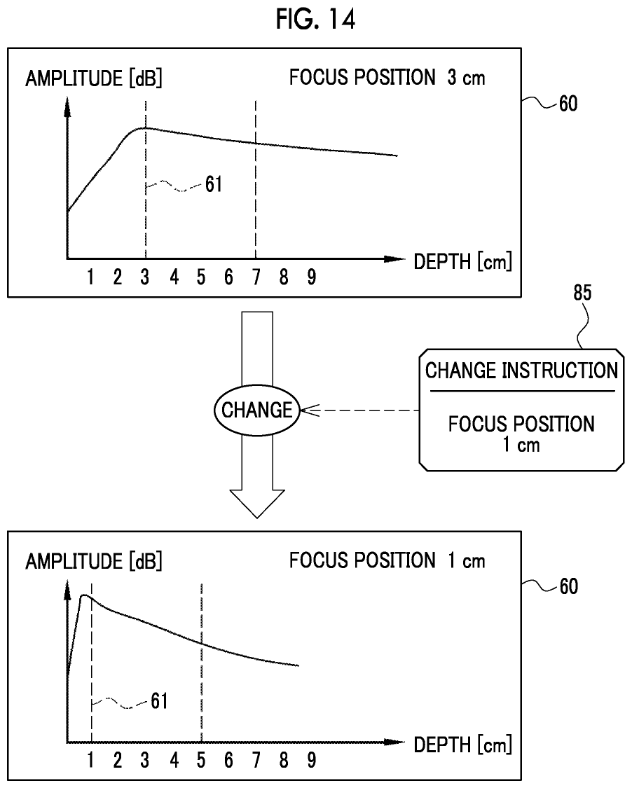

As shown in FIG. 14 as an example, the measurement condition may be only the focus position of the transmission wave TU. In this case, the operator OP inputs a change instruction 85 for the focus position through the input device 14. FIG. 14 shows an example in which the focus position is changed from 3 cm to 1 cm.

A plurality of reference signals 60 corresponding to each focus position are registered in the reference signal DB 56. The selection unit 67 reads out the reference signal 60 corresponding to the focus position of the change instruction 85, here, the focus position of 1 cm, from the reference signal DB 56, and outputs the reference signal 60 to the calculation unit 69. In addition, although not shown, the setting unit 68 outputs the focus position of the change instruction 85 to the transmission unit 31.

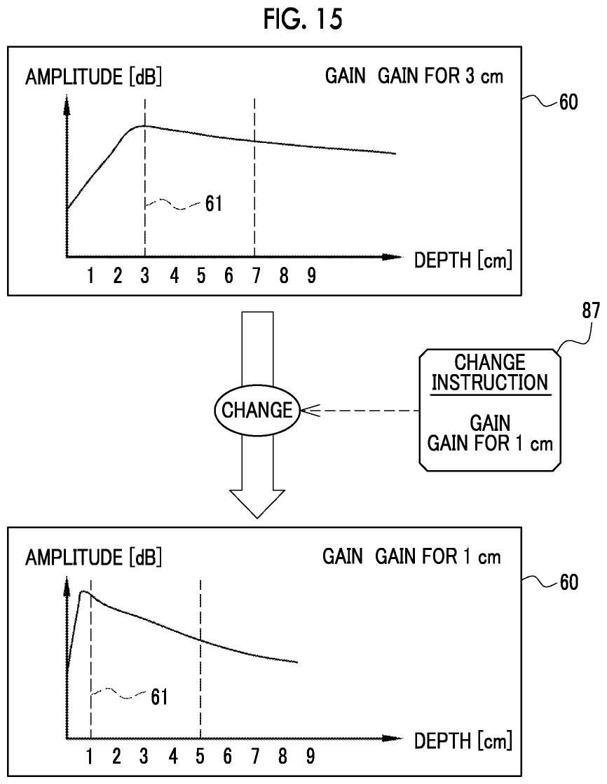

Similarly, as shown in FIG. 15 as an example, the measurement condition may be only the gain applied to the echo signal. In this case, the operator OP inputs a change instruction 87 for the gain through the input device 14. FIG. 15 shows an example in which the gain is changed from the gain for the focus position of 3 cm to the gain for the focus position of 1 cm.

A plurality of reference signals 60 corresponding to each gain are registered in the reference signal DB 56. The selection unit 67 reads out the reference signal 60 corresponding to the gain of the change instruction 87, here, the gain for the focus position of 1 cm, from the reference signal DB 56, and outputs the reference signal 60 to the calculation unit 69. In addition, although not shown, the setting unit 68 outputs the gain of the change instruction 87 to the reception unit 32.

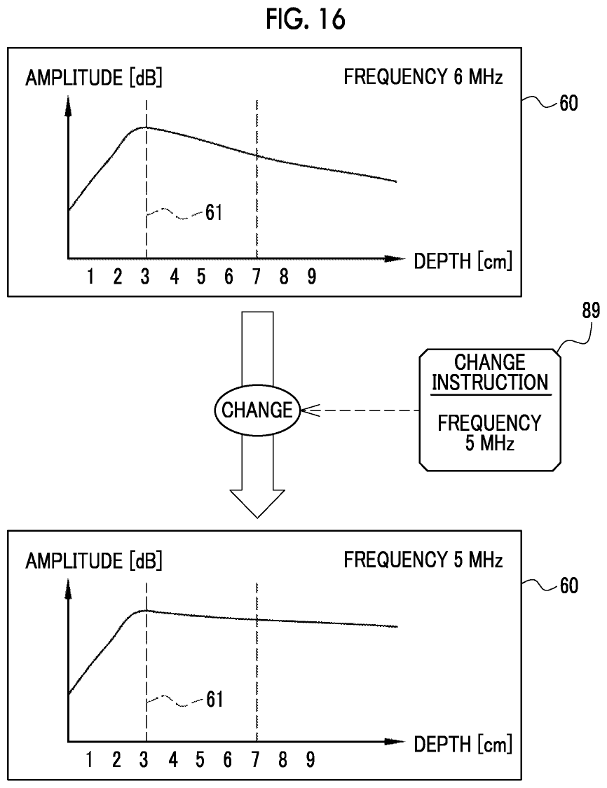

Alternatively, as shown in FIG. 16 as an example, the measurement condition may be only the frequency of the transmission wave TU. In this case, the operator OP inputs a change instruction 89 for the frequency through the input device 14. FIG. 16 shows an example in which the frequency is changed from 6 MHz to 5 MHz.

A plurality of reference signals 60 corresponding to each frequency are registered in the reference signal DB 56. The selection unit 67 reads out the reference signal 60 corresponding to the frequency of the change instruction 89, here, 5 MHz, from the reference signal DB 56, and outputs the reference signal 60 to the calculation unit 69. In addition, although not shown, the setting unit 68 outputs the frequency of the change instruction 89 to the transmission unit 31. The reference signal 60 having a frequency of 5 MHz has a smaller decrease rate in amplitude with increasing depth than the reference signal 60 having a frequency of 6 MHz. As described above, the measurement condition is not limited to all of the focus position of the transmission wave TU, the gain applied to the echo signal, and the frequency of the transmission wave TU, and may be at least one of these. Examples of a factor for changing any one of the focus position of the transmission wave TU, the gain applied to the echo signal, or the frequency of the transmission wave TU include the body type of the patient P, such as obesity and muscularity.

### Fourth Embodiment

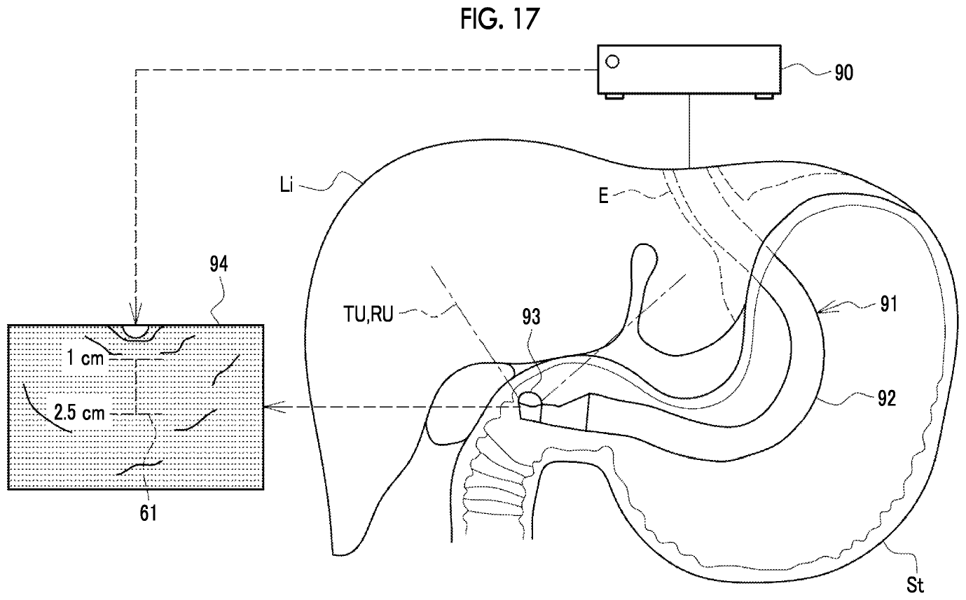

As shown in FIG. 17 as an example, an apparatus body 90 of the ultrasound diagnostic apparatus according to the present fourth embodiment can be connected to a body insertion probe 91 in addition to the body surface probe 12. The body insertion probe 91 is a so-called ultrasonic endoscope, and has a configuration in which an imaging unit (not shown) and a transducer array 93 are disposed at a distal end of an elongated insertion part 92. The insertion part 92 can be bent, and can change the orientation of the distal end. The transducer array 93 is composed of a plurality of transducers (not shown). The transducer array 93 includes, for example, a convex type, a radial type, and the like. In the convex type, the transducers are arranged in a curved shape to form a fan-shaped transmission wave TU. In the radial type, the transducers are disposed in a circular shape to form the transmission wave TU in all directions of 360 degrees. The convex type is suitable for observing the liver Li. In addition to the imaging unit and the transducer array 93, an acoustic lens or the like is built in the distal end of the body insertion probe 91.

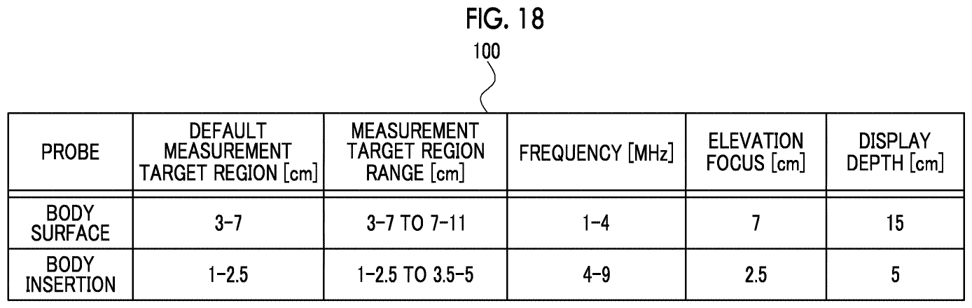

For example, in a case where the operator OP determines that the body type of the patient P is an obese type and it is difficult to measure the attenuation rate with the body surface probe 12, the operator OP uses the body insertion probe 91. The operator OP inserts the body insertion probe 91 into a mouth of the patient P. Then, the distal end of the body insertion probe 91 in which the transducer array 93 is disposed accesses the observation position of the liver Li along an esophagus E and further a stomach St. The operator OP inputs a transmission instruction of the transmission wave TU through the input device and transmits the transmission wave TU from the transducer array 93 toward the liver Li. A B-mode image 94 based on the reflected wave RU reflected in response to the transmission wave TU is displayed on the display. As indicated by a broken line 61 of the B-mode image 94 and as shown in a specification table 100 of FIG. 18 as an example, a default measurement target region in a case of the body insertion probe 91 is, for example, 1 cm to 2.5 cm. A range of the measurement target region is, for example, between 1 cm to 2.5 cm and 3.5 cm to 5 cm. A frequency of the transmission wave TU is, for example, 4 MHz to 9 MHz. In addition, the elevation focus is, for example, 2.5 cm, and the display depth is, for example, 5 cm. Note that the specification table 100 shows each value of the body surface probe 12 for comparison.

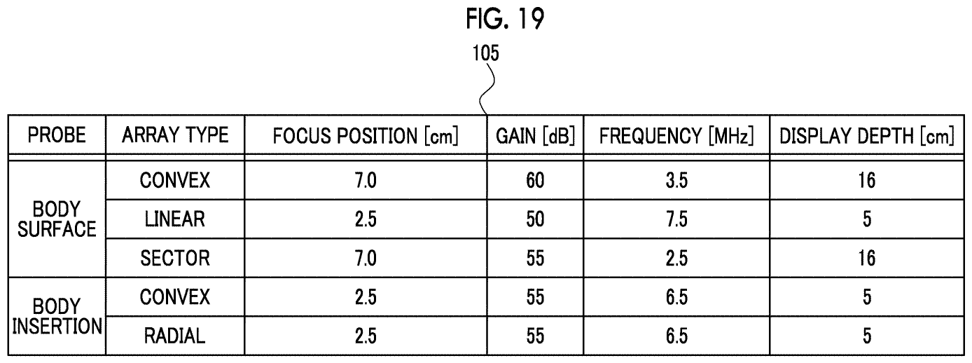

As shown in a specification table 105 of FIG. 19 as an example, representative values of the focus position of the transmission wave TU of the body insertion probe 91, the gain applied to the echo signal, the frequency of the transmission wave TU, and the display depth are substantially the same regardless of the type of the transducer array 93. The representative value is, for example, a maximum value or a median value. Note that, similarly to the specification table 100, the specification table 105 also shows each value of the body surface probe 12 for comparison.

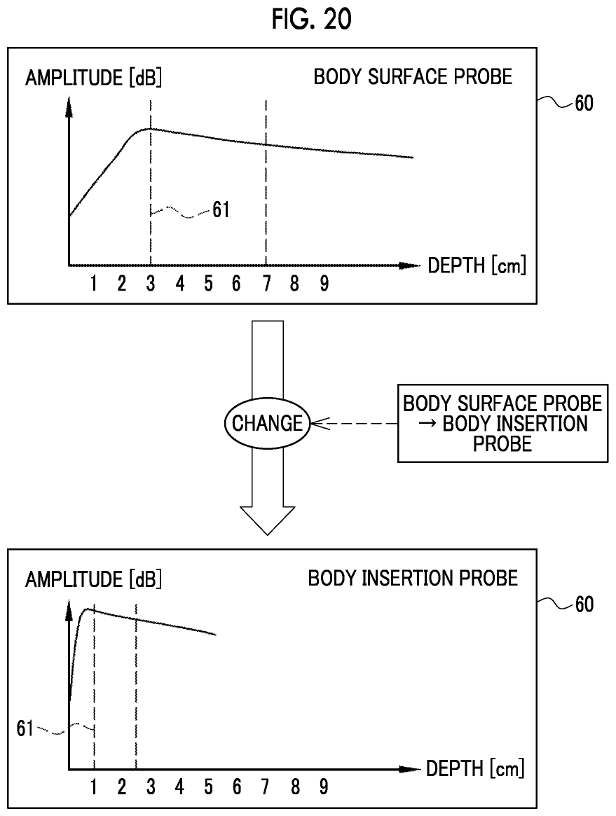

As shown in FIG. 20 as an example, in the reference signal DB 56, the reference signal 60 corresponding to the body insertion probe 91 is registered in addition to the reference signal 60 corresponding to the body surface probe 12. The reference signal 60 corresponding to the body insertion probe 91 has a measurement target region of 1 cm to 2.5 cm by default. In addition, the reference signal 60 corresponding to the body insertion probe 91 has an amplitude that gradually decreases in the measurement target region of 1 cm to 2.5 cm. Here, only one reference signal 60 corresponding to the body insertion probe 91 is drawn, but in practice, a plurality of reference signals 60 corresponding to the range of the measurement target region of the specification table 100 shown in FIG. 18, and the focus position of the transmission wave TU, the gain applied to the echo signal, and the frequency of the transmission wave TU of the specification table 105 shown in FIG. 19 are prepared.

In a case where the body insertion probe 91 is connected to the apparatus body 90 instead of the body surface probe 12, the selection unit 67 reads out the reference signal 60 corresponding to the body insertion probe 91 from the reference signal DB 56 and outputs the readout reference signal 60 to the calculation unit 69. In addition, although not shown, the setting unit 68 outputs the setting information 63 corresponding to the body insertion probe 91 to the transmission unit 31 and the reception unit 32. As described above, the measurement condition may be the type of the probe. Even in a case where the type of the probe is changed, it is possible to suppress a decrease in the calculation accuracy of the attenuation rate of the measurement signal 62. In addition, in a case where the type of the probe is the body surface probe 12 and the body insertion probe 91, it is possible to cover all conceivable types.

The reference signal 60 corresponding to the model name and/or the model number of the body insertion probe 91 may be selected. The reference signal 60 corresponding to the type of the transducer array 93, such as a convex type or a radial type, may be selected. The reference signal 60 corresponding to the organ (for example, the liver or the pancreas) observed by the body insertion probe 91 may be selected. In these cases, the model name and/or the model number of the body insertion probe 91, the type of the transducer array 93, or the organ observed by the body insertion probe 91 is input by the operator OP through the input device 14. Alternatively, the model name and/or the model number of the body insertion probe 91, the type of the transducer array 93, or the organ observed by the body insertion probe 91 may be acquired by reading the model name and/or the model number, the type, or the organ from a radio frequency identification (RFID) tag or the like attached to a connector of the body insertion probe 91 connected to the apparatus body 90. Further, the organ to be observed may be estimated from the ultrasound image, and the reference signal 60 corresponding to the estimation result may be selected.

### Fifth Embodiment

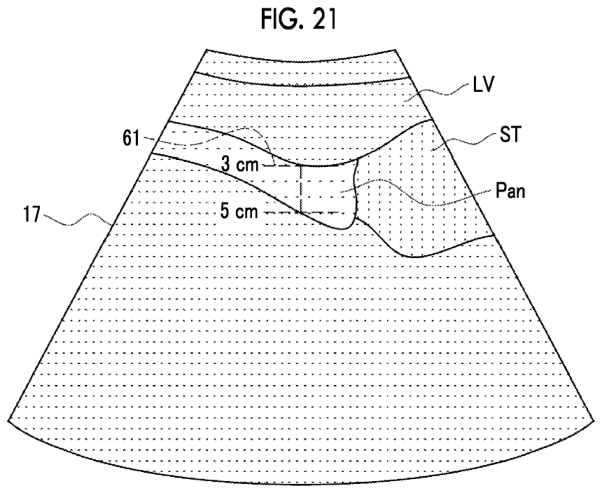

In each of the above-described embodiments, the liver Li is exemplified as the measurement target organ of the attenuation rate, but the present disclosure is not limited thereto. As shown in FIG. 21 as an example, a pancreas Pan may be used as the measurement target organ of the attenuation rate. The default measurement target region in a case of the pancreas Pan is 3 cm to 5 cm as shown in the drawing. The range of 3 cm to 5 cm is based on a general depth of the pancreas Pan from the body surface. It should be noted that, although the case of the body surface probe 12 is shown in FIG. 21, the attenuation rate of the pancreas Pan may be measured using the body insertion probe 91.

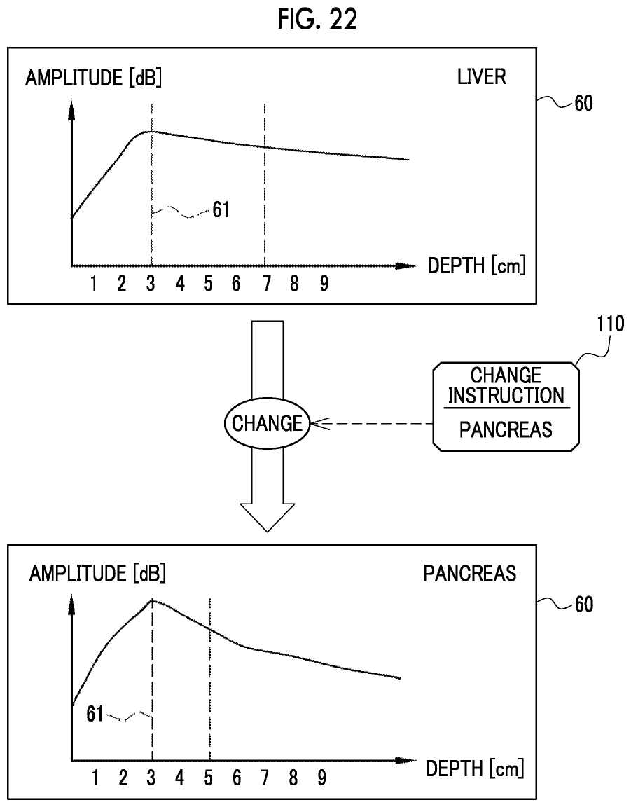

As shown in FIG. 22 as an example, in addition to the reference signal 60 corresponding to the liver Li, a reference signal 60 corresponding to the pancreas Pan is registered in the reference signal DB 56. In the reference signal 60 corresponding to the pancreas Pan, the measurement target region is 3 cm to 5 cm by default as described above. In the reference signal 60 corresponding to the pancreas Pan, the amplitude gradually decreases in the measurement target region of 3 cm to 5 cm. Here, only one reference signal 60 corresponding to the pancreas Pan is drawn, but in practice, a plurality of reference signals 60 corresponding to a range of the measurement target region that can be set, a focus position of the transmission wave TU, a gain applied to the echo signal, and a frequency of the transmission wave TU are prepared.

The operator OP inputs a change instruction 110 for the measurement target organ through the input device 14. In a case where the change instruction 110 to change the measurement target organ to the pancreas Pan instead of the liver Li is input, the selection unit 67 reads out the reference signal 60 corresponding to the pancreas Pan from the reference signal DB 56 and outputs the reference signal 60 to the calculation unit 69. In addition, although not shown, the setting unit 68 outputs the setting information 63 corresponding to the pancreas Pan to the transmission unit 31 and the reception unit 32. As described above, the measurement condition may be the measurement target organ. It is possible to suppress a decrease in calculation accuracy of the attenuation rate of the measurement signal 62 even in a case where the measurement target organ is changed. In addition, in a case where the measurement target organ is the liver Li and the pancreas Pan, it is possible to cover all conceivable measurement target organs.

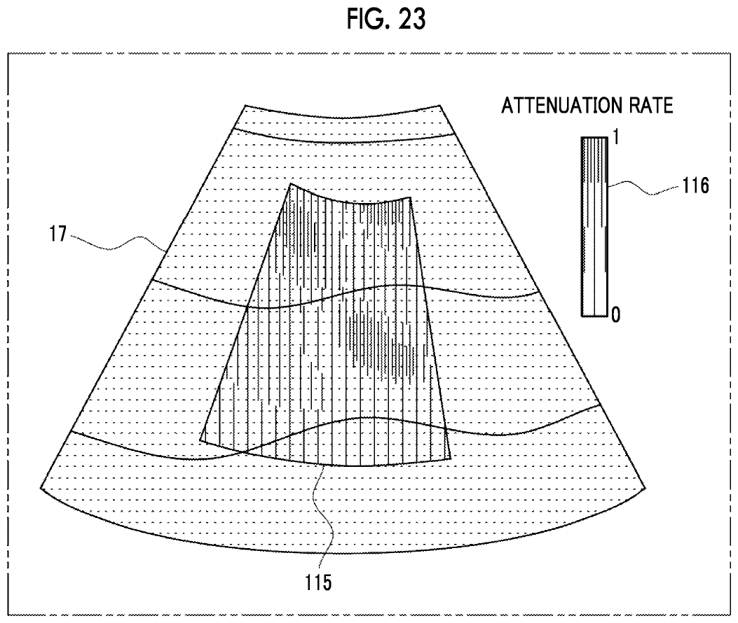

In FIG. 11, an example in which the calculation result 71 itself is displayed as the calculation result information of the attenuation rate is shown, but the present disclosure is not limited thereto. As shown in FIG. 23 as an example, a color map 115 in which the level of the attenuation rate is represented by a color may be displayed as the calculation result information of the attenuation rate. The color map 115 is generated by calculating the attenuation rate for each divided region obtained by subdividing the corresponding region and filling each divided region with a color corresponding to the level of the calculated attenuation rate.

The image generation unit 33 superimposes the color map 115 on the B-mode image 17. In addition, the image generation unit 33 displays a color bar 116 indicating the level of the attenuation rate represented by the color of the color map 115, on a side of the B-mode image 17. In this manner, the operator OP can visually ascertain the level of the attenuation rate. The operator OP can determine whether or not the calculation result 71 of the attenuation rate is appropriate. The calculation result 71 itself may be displayed together with the color map 115. In addition, although the B-mode image 17 of the body surface probe 12 is shown in FIG. 23, the color map 115 may be superimposed on the B-mode image 94 of the body insertion probe 91 shown in FIG. 17.

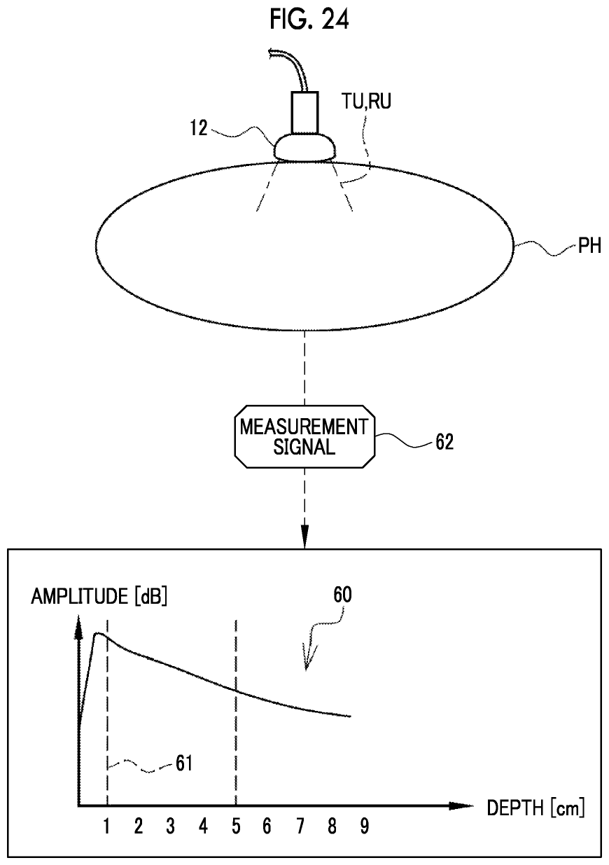

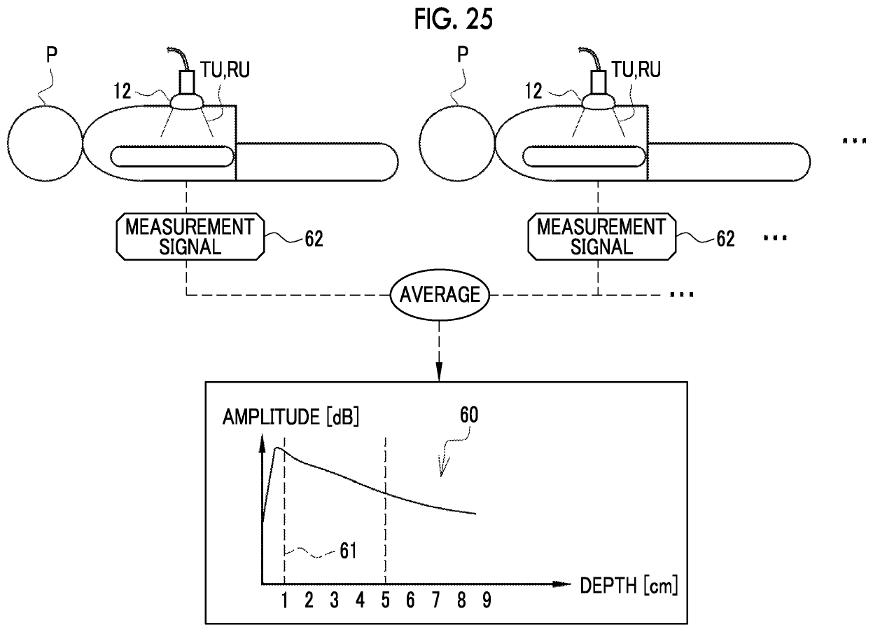

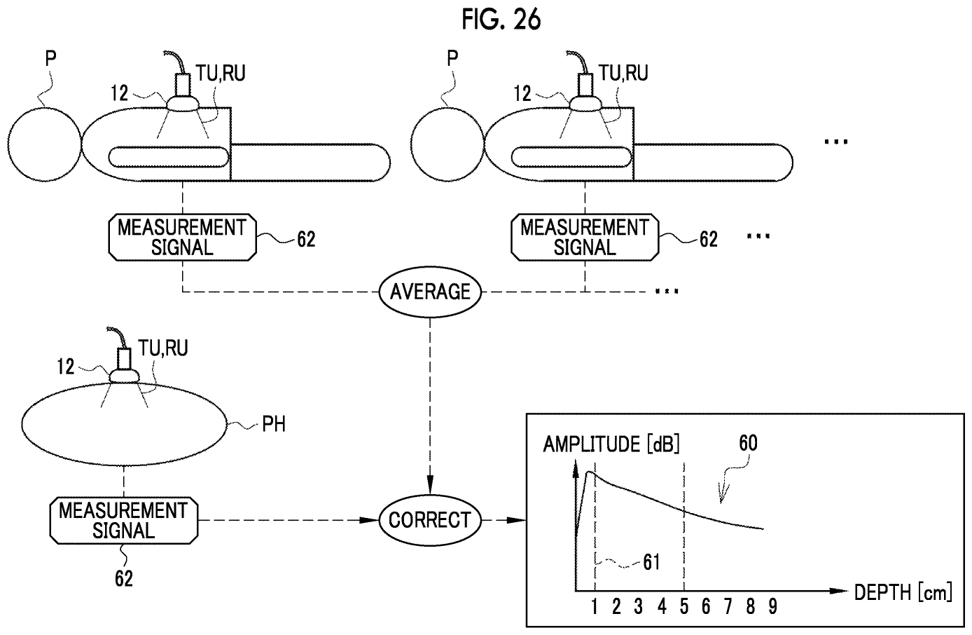

As shown in FIG. 24 as an example, the reference signal 60 is a measurement signal 62 obtained by irradiating a phantom PH imitating a human body with a transmission wave TU having a focus position and a frequency corresponding to the measurement target region. Alternatively, as shown in FIG. 25 as an example, the reference signal 60 may be an average value of the measurement signals 62 of a plurality of representative patients P. In addition, as shown in FIG. 26 as an example, the reference signal 60 may be obtained by correcting the measurement signal 62 obtained by using the phantom PH with the average value of the measurement signals 62 of the plurality of representative patients P. The average value is obtained by adding the measurement signals 62 of the plurality of representative patients P per unit depth and dividing the added value by the number of the patients P. The average value is an example of a “representative value” according to the technology of the present disclosure. A median value, a mode value, or the like may be used as the representative value.

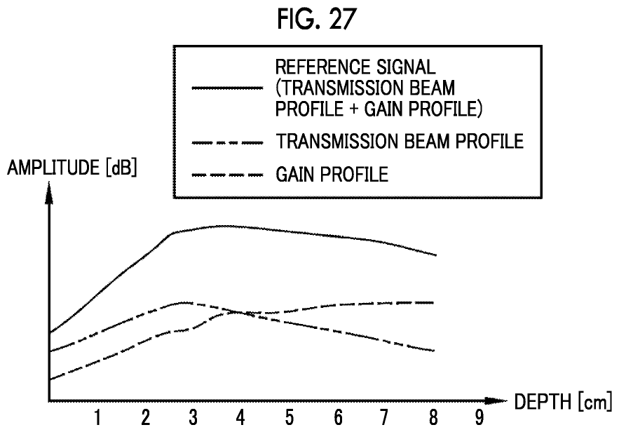

Further, the reference signal 60 may be estimated based on a theoretical model. In a case of being estimated based on the theoretical model, the reference signal 60 may be generated based on a transmission beam profile and a gain profile. As the simplest method, as shown in FIG. 27 as an example, a method of generating the reference signal 60 by adding the transmission beam profile and the gain profile is considered. The transmission beam profile is beam forming data dependent on the transmission focus position extracted from the measurement signal 62. In addition, the gain profile is beam forming data obtained by gain correction.

In each of the above-described embodiments, for example, each process of each processing unit, such as the image generation unit 33, the signal acquisition unit 65, the instruction receiving unit 66, the selection unit 67, the setting unit 68, the calculation unit 69, and the detection unit 80, is executed by any computer. In addition, any computer may execute these processes using a processor as hardware, a program as software, or a combination thereof. In such a case, the processor is configured to execute various types of processes in each of the above-described embodiments in cooperation with the program, and may function as each unit or each means in each of the above-described embodiments. Additionally, the execution order of the process by the processor is not limited to the order described above and may be changed as appropriate. Any computer may be a general-purpose computer, a special-purpose computer, a workstation, or other system capable of executing each process.

The processor may be configured with one or a plurality of pieces of hardware, and the type of hardware is not limited. For example, the processor may be configured with hardware such as the exemplified CPU 51, a micro processing unit (MPU), a programmable logic device such as a field-programmable gate array (FPGA), a dedicated circuit for executing a specific process such as an application-specific integrated circuit (ASIC), a graphics processing unit (GPU), or a neural processing unit (NPU). Additionally, the type of hardware may be a combination of different types of hardware. In a case where a plurality of hardware components are configured to execute one or a plurality of processes of a certain processor, the plurality of hardware components may be present in devices physically separate from each other or may be present within the same device. Further, in any of the embodiments, the order of each process performed by the processor is not limited to the order described above, and may be changed as appropriate. In addition, the hardware is implemented in a form of an electric circuit (circuitry) in which circuit elements such as semiconductor elements are combined.

Further, the program may be firmware or software such as a microcode. In addition, the program may be, for example, a program module group, and each function thereof may be implemented by a processor configured to execute each function. The program may be a program code or a plurality of code segments stored in one or a plurality of non-transitory computer-readable media (for example, a storage medium or other storage). The program may be divided and stored in the plurality of non-transitory computer-readable media present in devices physically separate from each other. The program code or code segment may represent any combination of a procedure, a function, a subprogram, a routine, a subroutine, a module, a software package, a class, or an instruction, a data structure, or a program statement. The program code or code segment may be connected to another code segment or a hardware circuit by transmitting and receiving information, data, an argument, a parameter, or a content of a memory.

The technology according to the following supplementary notes can be understood from the above description.

### Supplementary Note 1

An ultrasound diagnostic apparatus comprising:
a processor,
in which the processor is configured to:
   select a reference signal corresponding to a measurement condition from among a plurality of reference signals; and
   calculate, based on a measurement signal obtained by measuring a subject and the selected reference signal, an attenuation rate of the measurement signal.

### Supplementary Note 2

The ultrasound diagnostic apparatus according to Supplementary Note 1,
in which the measurement condition is at least one of a measurement target region, a focus position of a transmission wave, a gain applied to an echo signal, a frequency of the transmission wave, a type of a probe, or a measurement target organ.

### Supplementary Note 3

The ultrasound diagnostic apparatus according to Supplementary Note 1,
in which the measurement condition is at least one of a measurement target region, a focus position of a transmission wave, a gain applied to an echo signal, or a frequency of the transmission wave.

### Supplementary Note 4

The ultrasound diagnostic apparatus according to Supplementary Note 2 or 3,
in which the processor is configured to:
   detect the measurement target region satisfying a detection condition; and
   select a reference signal corresponding to the detected measurement target region.

### Supplementary Note 5

The ultrasound diagnostic apparatus according to Supplementary Note 4,
in which the detection condition includes a condition related to a distance to the measurement target organ and a condition related to an intensity of the echo signal.

### Supplementary Note 6

The ultrasound diagnostic apparatus according to Supplementary Note 2,
in which the type of the probe is any of a body surface probe or a body insertion probe.

### Supplementary Note 7

The ultrasound diagnostic apparatus according to Supplementary Note 2,
in which the measurement target organ is any of a liver or a pancreas.

### Supplementary Note 8

The ultrasound diagnostic apparatus according to any one of Supplementary Notes 1 to 7,
in which calculation result information of the attenuation rate is displayed on a display unit.

### Supplementary Note 9

The ultrasound diagnostic apparatus according to Supplementary Note 8,
in which the calculation result information is a color map in which a level of the attenuation rate is represented by a color.

### Supplementary Note 10

The ultrasound diagnostic apparatus according to Supplementary Note 8 or 9,
in which the reference signal used for calculating the attenuation rate is displayed on the display unit together with the calculation result information.

### Supplementary Note 11

The ultrasound diagnostic apparatus according to any one of Supplementary Notes 1 to 10,
in which the reference signal is any of a measurement signal obtained by using a phantom, a representative value of measurement signals of a plurality of representative subjects, or a measurement signal obtained by using a phantom, which is corrected by the representative value of the measurement signals of the plurality of representative subjects.

### Supplementary Note 12

The ultrasound diagnostic apparatus according to any one of Supplementary Notes 1 to 10,
in which the reference signal is generated based on a transmission beam profile and a gain profile.

The technology of the present disclosure can also appropriately combine various embodiments and/or various modification examples described above. It goes without saying that the present disclosure is not limited to each of the embodiments described above, and various configurations can be employed without departing from the gist. Furthermore, the technology of the present disclosure extends to a storage medium that non-transitorily stores the program, and a computer program product including the program, in addition to the program.

The described content and the illustrated content above are detailed descriptions of portions related to the technology of the present disclosure and are merely an example of the technology of the present disclosure. For example, the above description of the configuration, the function, the action, and the effect are the description of examples of the configuration, the function, the action, and the effect of the parts according to the technology of the present disclosure. Accordingly, it goes without saying that unnecessary parts may be deleted, new elements may be added, or replacements may be made with respect to the described content and the illustrated content above within a range that does not depart from the gist of the technology of the present disclosure. Further, in order to avoid complication and facilitate understanding of the portions according to the technology of the present disclosure, the description related to common general knowledge not requiring special description in order to implement the technology of the present disclosure is omitted in the described content and the illustrated content above.

In the present specification, “A and/or B” is synonymous with “at least one of A or B”. That is, “A and/or B” means that it may be only A, only B, or a combination of A and B. In addition, in the present specification, in a case where three or more matters are expressed by being connected by “and/or”, the same concept as “A and/or B” is applied.

All documents, patent applications, and technical standards described in the present specification are incorporated in the present specification by reference to the same extent as in a case where each of the documents, the patent applications, and the technical standards are specifically and individually indicated to be incorporated by reference. Explanation of References

What is claimed is:

1. An ultrasound diagnostic apparatus comprising:
a processor,
wherein the processor is configured to:
select a reference signal corresponding to a measurement condition from among a plurality of reference signals; and
calculate, based on a measurement signal obtained by measuring a subject and the selected reference signal, an attenuation rate of the measurement signal.

2. The ultrasound diagnostic apparatus according to claim 1,
wherein the measurement condition is at least one of a measurement target region, a focus position of a transmission wave, a gain applied to an echo signal, a frequency of the transmission wave, a type of a probe, or a measurement target organ.

3. The ultrasound diagnostic apparatus according to claim 1,
wherein the measurement condition is at least one of a measurement target region, a focus position of a transmission wave, a gain applied to an echo signal, or a frequency of the transmission wave.

4. The ultrasound diagnostic apparatus according to claim 2,
wherein the processor is configured to:
detect the measurement target region satisfying a detection condition; and
select a reference signal corresponding to the detected measurement target region.

5. The ultrasound diagnostic apparatus according to claim 4,
wherein the detection condition includes a condition related to a distance to the measurement target organ and a condition related to an intensity of the echo signal.

6. The ultrasound diagnostic apparatus according to claim 2,
wherein the type of the probe is any of a body surface probe or a body insertion probe.

7. The ultrasound diagnostic apparatus according to claim 2,
wherein the measurement target organ is any of a liver or a pancreas.

8. The ultrasound diagnostic apparatus according to claim 1,
wherein calculation result information of the attenuation rate is displayed on a display unit.

9. The ultrasound diagnostic apparatus according to claim 8,
wherein the calculation result information is a color map in which a level of the attenuation rate is represented by a color.

10. The ultrasound diagnostic apparatus according to claim 8,
wherein the reference signal used for calculating the attenuation rate is displayed on the display unit together with the calculation result information.

11. The ultrasound diagnostic apparatus according to claim 1,
wherein the reference signal is any of a measurement signal obtained by using a phantom, a representative value of measurement signals of a plurality of representative subjects,
or a measurement signal obtained by using a phantom, which is corrected by the representative value of the measurement signals of the plurality of representative subjects.

12. The ultrasound diagnostic apparatus according to claim 1,
wherein the reference signal is generated based on a transmission beam profile and a gain profile.

13. An operation method of an ultrasound diagnostic apparatus, comprising:
selecting a reference signal corresponding to a measurement condition from among a plurality of reference signals; and
calculating, based on a measurement signal obtained by measuring a subject and the selected reference signal, an attenuation rate of the measurement signal.

14. A non-transitory computer-readable storage medium storing an operation program of an ultrasound diagnostic apparatus, the operation program causing a computer to execute a process comprising:
selecting a reference signal corresponding to a measurement condition from among a plurality of reference signals; and
calculating, based on a measurement signal obtained by measuring a subject and the selected reference signal, an attenuation rate of the measurement signal.
